(19) United States
(12) Patent Application Publication
(10) Pub. No.: US 2026/0114782 A1
(43) Pub. Date: Apr. 30, 2026

(54) System and method for virtual cardiac ischemia imaging

(71) Applicant: National Central University, Taoyuan City (TW)

(72) Inventors: Tse-Yu Lin, New Taipei City (TW); Chen Lin, Taipei City (TW); Men-Tzung Lo, Tainan City (TW)

(21) Appl. No.: 18/925,081

(22) Filed: Oct. 24, 2024

(51) Int. Cl. A61B 5/341; A61B 5/00

(52) U.S. Cl. CPC A61B 5/341; A61B 5/6823; A61B 5/684

(57) ABSTRACT

A system for virtual cardiac ischemia imaging comprises a positioning device, a probe sensor device, a lead electrode sensor device, an analysis device, and a display device. Through the system for virtual cardiac ischemia imaging, lead electrodes is precisely attached on body surface of torso of a subject in need thereof, ischemic condition of heart of the subject is precisely simulated and does not require utilization of nuclear medicine examination to estimate shape of the heart of the subject. A method for virtual cardiac ischemia imaging is realized through the components of the system for virtual cardiac ischemia imaging.

### BACKGROUND OF THE INVENTION

### 1. Field of the Invention

The present invention is related to virtual cardiac ischemia imaging technique, in particular related to system and method for virtual cardiac ischemia imaging.

### 2. Description of the Prior Art

Present electrocardiographic imaging technique usually requires accurate attachment of lead electrodes one-by-one on desired positions of a subject in need thereof, so as to precisely observe ischemic regions of heart of the subject. However, human torso varies in size and shape with respect to factors such as gender and body type, accuracy for attachment of lead electrodes to a subject is affected by skill of medical staff operating virtual cardiac ischemia imaging, and simulation of ischemic condition of heart of the subject is therefore prone to human error during attachment of lead electrodes. In addition, before electrocardiographic imaging is conducted on a subject, nuclear medicine examination of heart on the subject is usually required for estimating position and shape of heart within torso of the subject to avoid human error during attachment of the lead electrodes. The utilization of nuclear medicine examination puts the subject under exposure to radiation and may result in unexpected radiation damage.

Therefore, there is an unmet need in the field to provide a system and a method for virtual cardiac ischemia imaging to accurately attach lead electrodes onto a subject, precisely simulate ischemic condition of heart of the subject and free from utilization of nuclear medicine examination.

### SUMMARY OF THE INVENTION

A system for virtual cardiac ischemia imaging comprises a positioning device, a probe sensor device, a lead electrode sensor device, an analysis device, and a display device. The probe sensor device is coupled with the positioning device. The lead electrode sensor device is coupled with the positioning device. The analysis device is coupled to the probe sensor device and the lead electrode sensor device. The display device is coupled to the analysis device. The positioning device is configured to position an attachment position and establish a coordinate system for a subject in need thereof. The probe sensor device is configured to acquire a relative position from body surface to heart of the subject and heart topology for the heart of the subject. The lead electrode sensor device is configured to acquire electrocardiogram signal data for the subject from the attachment position. The analysis device is configured to establish an established digital twin of the heart according to an initial digital twin of a heart model, a heart coordinate according to the relative position within the coordinate system, the heart topology and the coordinate system, label a reference ischemic region on the established digital twin of the heart, simulate ischemic reference signal data according to the reference ischemic region for the established digital twin of the heart, perform pre-processing on the electrocardiogram signal data, extract first feature vector from the electrocardiogram signal data, extract second feature vector from the ischemic reference signal data, determine heart ischemic region of the heart and heart ischemic level of the heart ischemic region according to the first feature vector and the second feature vector, and label the heart ischemic region and the heart ischemic level of the heart ischemic region on the established digital twin of the heart. The display device is configured to display the heart ischemic region and the heart ischemic level of the heart ischemic region on the established digital twin of the heart on a two-dimensional plane.

A method for virtual cardiac ischemic imaging comprising a positioning device positioning an attachment position and establishing a coordinate system for a subject in need thereof; a probe sensor device acquiring a relative position from body surface to heart of the subject and heart topology for the heart of the subject; a lead electrode sensor device acquiring electrocardiogram signal data for the subject from the attachment position; an analysis device establishing an established digital twin of the heart according to an initial digital twin of a heart model, a heart coordinate according to the relative position within the coordinate system, the heart topology and the coordinate system; the analysis device labeling a reference ischemic region on the established digital twin of the heart; the analysis device simulating ischemic reference signal data according to the reference ischemic region for the established digital twin of the heart; the analysis device performing pre-processing on the electrocardiogram signal data; the analysis device extracting first feature vector from the electrocardiogram signal data; the analysis device extracting second feature vector from the ischemic reference signal data; the analysis device determining a heart ischemic region of the heart and a heart ischemic level of the heart ischemic region according to the first feature vector and the second feature vector; the analysis device labeling the heart ischemic region and the heart ischemic level of the heart ischemic region on the established digital twin of the heart; and a display device displaying the heart ischemic region and the heart ischemic level of the heart ischemic region on the established digital twin of the heart on a two-dimensional plane.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is an exemplary diagram illustrating a system for virtual cardiac ischemia imaging.

FIG. 2 is a schematic diagram illustrating assembling relationships between the positioning device, the lead electrode sensor device and the probe sensor device with respect to torso of a subject.

FIG. 3A and FIG. 3B are schematic diagrams illustrating detailed configurations of the positioning device.

FIG. 4A and FIG. 4B are schematic diagrams illustrating detailed configurations of the lead electrode sensor device.

FIG. 5 is a flow diagram illustrating steps to compute an established digital twin of the heart of the subject and ischemic reference signal data for the established digital twin of the heart for virtual cardiac ischemia imaging.

FIG. 6 is a flow diagram illustrating steps to determine ischemic condition of the subject using the established digital twin of the heart of the subject and the electrocardiogram signal data acquired by the lead electrode sensor device during virtual cardiac ischemia imaging.

FIGS. 7A to 7D are schematic diagrams of 11 basic regions corresponding to anatomical areas of the heart of the subject.

FIGS. 8A and 8B are schematic diagrams illustrating an established digital twin of the heart being projected on a two-dimensional plane.

### DETAILED DESCRIPTION

Below, exemplary embodiments will be described in detail with reference to accompanying drawings so as to be easily realized by a person having ordinary knowledge in the art. The inventive concept may be embodied in various forms without being limited to the exemplary embodiments set forth herein. Descriptions of well-known parts are omitted for clarity, and like reference numerals refer to like elements throughout.

As used herein, when describing an object “comprises,” “includes” or “has” a limitation, unless otherwise specified, it may additionally encompass other elements, structures, regions, parts, apparatus, devices, systems, steps, connections, modules, units, etc., and should not exclude others. Further, unless otherwise specified, wordings in singular forms such as “a,” “an” and “the” also pertain to plural forms, and wordings such as “or” and “and/or” may be used interchangeably.

As used herein, the phrase “at least one,” in reference to a list of one or more elements, should be understood to mean at least one element selected from any one or more of the elements in the list of elements, but not necessarily including at least one of each and every element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently, “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

As used herein, the terms “subject in need thereof,” “subject,” and “patient” may be used interchangeably.

As used herein, the term “hingedly connected” refer to a socket and ball connection, a socket and spigot connection, or the like.

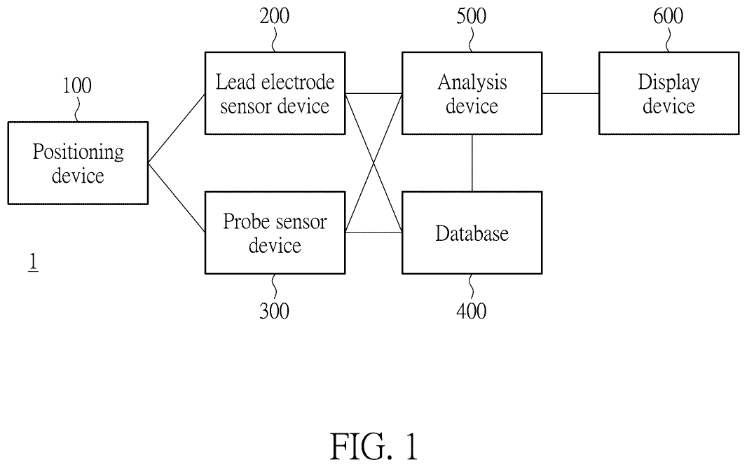

FIG. 1 is an exemplary diagram illustrating a system 1 for virtual cardiac ischemia imaging, where the system 1 may include a positioning device 100, a lead electrode sensor device 200, a probe sensor device 300, a database 400, an analysis device 500 and a display device 600. The elements of the system 1 may be coupled to each other via any suitable wired or wireless manner.

The positioning device 100 may be used to position an attachment position and establish a coordinate system for a subject of virtual cardiac ischemia imaging. The attachment position is on body surface of torso of the subject. The coordinate system defines a three-dimensional space having the torso of the subject with respect to arrangement of the positioning device 100.

The lead electrode sensor device 200 may be coupled to the positioning device 100 and may be used to acquire electrocardiogram signal data for the subject from the attachment position at body surface of the subject. The electrocardiogram signal data may be analyzed through body surface potential mapping by the analysis device 500 to determine ischemic condition of the heart of the subject.

The probe sensor device 300 may be coupled to the positioning device 100 and may be used to acquire relative position from body surface to heart of the subject and heart topology for the heart of the subject according to the coordinate system.

The database 400 may be coupled to the lead electrode sensor device 200, the probe sensor device 300 and the analysis device 500, and may be used to store the coordinate system established by the positioning device 100, the electrocardiogram signal data acquired by the lead electrode sensor device 200, an initial digital twin of a heart model, the relative position from body surface to the heart and the heart topology acquired by the probe sensor device 300, an established digital twin of the heart computed from the initial digital twin of the heart model, the heart coordinate and the heart topology by the analysis device 500, the ischemic reference signal of the heart simulated by the analysis device 500, and an ischemic condition computed from the established digital twin of the heart and the electrocardiogram signal data by the analysis device 500.

The analysis device 500 may be coupled to the lead electrode sensor device 200, the probe sensor device 300 and the database 400, and may be used to compute established digital twin of the heart according to the initial digital twin of the heart model, the coordinate system, the heart coordinate and the heart topology, and compute ischemic condition of the heart of the subject according to the established digital twin of the heart and the electrocardiogram signal data. The ischemic condition may include heart ischemic region and heart ischemic level of the heart ischemic region.

The display device 600 may be coupled to the analysis device 500 and may be used to display the ischemic condition of the heart of the subject using the established digital twin of the heart.

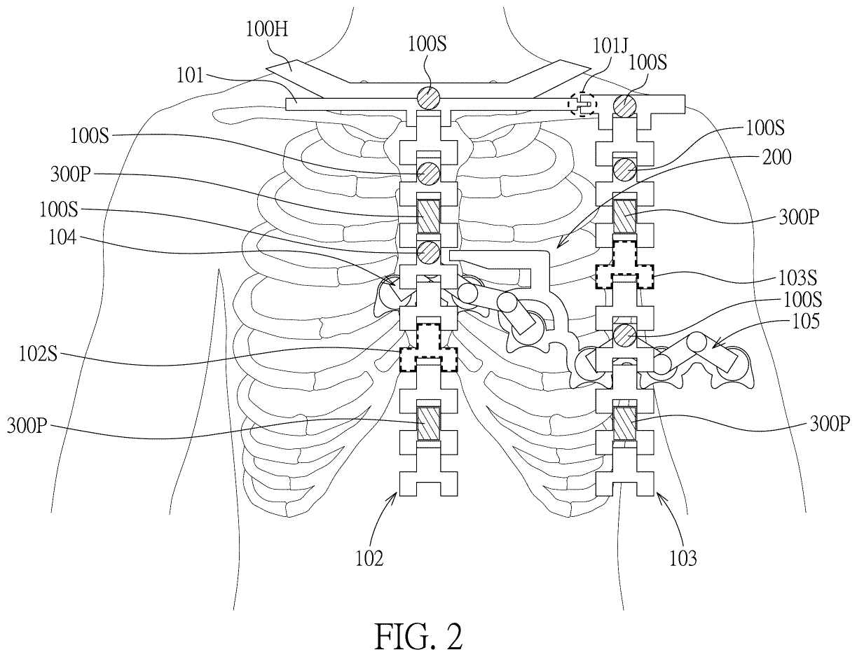

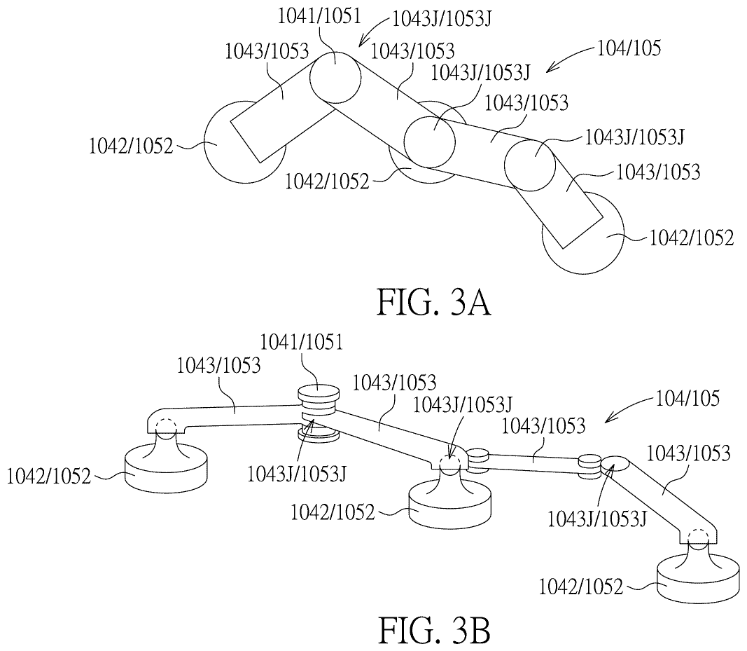

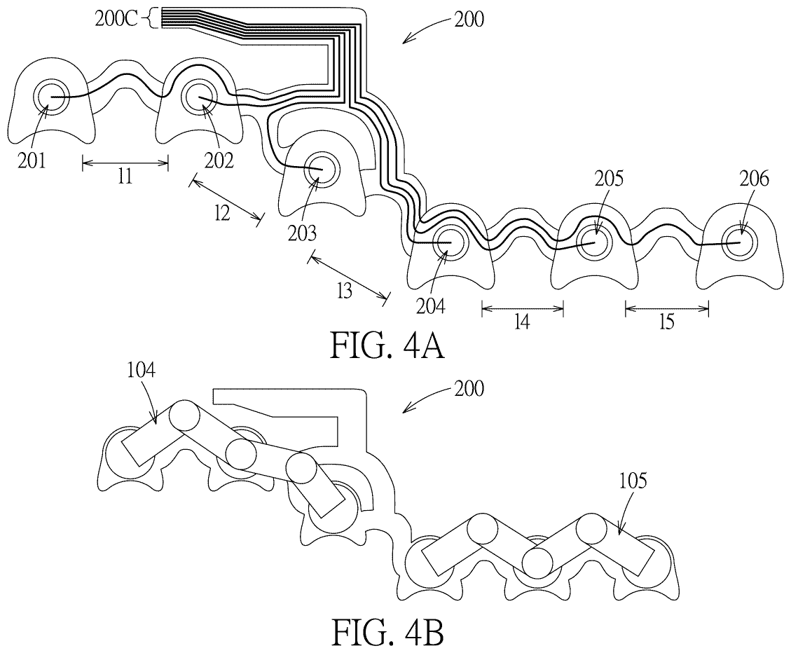

FIG. 2, FIG. 3A, FIG. 3B, FIG. 4A and FIG. 4B are schematic diagrams illustrating detailed configurations of the positioning device 100, the lead electrode sensor device 200 and the probe sensor device 300 of the system 1. FIG. 2 is used to depict assembling relationships between the positioning device 100, the lead electrode sensor device 200 and the probe sensor device 300 with respect to the torso of the subject. FIG. 3A and FIG. 3B are used to depict detailed configurations of lead electrode supports of the positioning device 100. FIG. 4A and FIG. 4B are used to depict detailed configurations of the lead electrode sensor device 200.

The positioning device 100 may include a hanger support 100H, a main support 101, branch supports 102 and 103, and lead electrode supports 104 and 105. The hanger support 100H may be used to hang the positioning device 100 on the torso of the subject, such as have the hanger support 100H wore around neck of the subject, thereby to determine attachment positions for the lead electrode sensor device 200. When viewed from standing posture of the subject, the main support 101 is horizontally placed against the torso, the branch supports 102 and 103 are vertically placed against the torso, and the lead electrode supports 104 and 105 are fixed on the branch supports 102 and 103, respectively.

When placing the positioning device 100 on the subject in a standing posture, the arrangement of main support 101, the branch supports 102 and 103, and the lead electrode supports 104 and 105 may be described as follows: the main support 101 is placed along longitudinal direction of left clavicle of the subject; the branch supports 102 and 103 are hingedly connected to the main support 101, where the branch support 102 (first branch support) may be placed along longitudinal direction of sternum of the subject and the branch support 103 (second branch support) may be placed along mid-clavicular line of the left clavicle of the subject; the lead electrode support 104 (first lead electrode support) may be removably connected to the branch support 103 and may branch out on from the branch support 102 to torso surface (e.g., in the chest area) of the subject; and the lead electrode support 105 (second lead electrode support) may branch out on from the branch support 103 to torso surface (e.g., in the chest area) of the subject. FIG. 2 only shows two sets of lead electrode supports 104 and 105, but other sets of lead electrode supports may also be present according to required configurations of the lead electrode sensor device 200.

Moreover, the main support 101 may include at least one joint 101J, the branch support 102 may be made of at least two sections 102S (first sections) hingedly concatenated in a row and the branch support 103 may be made of at least two sections 103S (second sections) hingedly concatenated in a row. Therefore, the main support 101 and the branch supports 102 and 103 may be hingedly adjusted to enable adjustment of the positioning device 100 according to variations of body types. For example, if the subject has conditions of thoracic scoliosis, the sections 102S of the branch support 102 and sections 103S if the branch support 103 may be hingedly adjusted to be fully conform to curved shapes of the sternum and mid-clavicular line of the left clavicle of the subject, respectively. In another example, if the left clavicle of the subject has an unusual shape than average individuals, the main support 101 may be adjusted by the at least one joint 101J to be fully conform to shape of the left clavicle of the subject.

Furthermore, each set of the lead electrode support 104/105 may include one connection knot 1041/1051, three attachment disks 1042/1052, four support sections 1043/1053, and three adjustment joints 1043J/1053J. The connection knot 1041/1051 may be used to fix the lead electrode support 104/105 onto desired positions on the corresponding branch support 102/103 (e.g., one of the sections 102S/103S). The attachment disks 1042/1052 may be realized as suction disks or adhesive disks and may be used to assist attachment of the lead electrode sensor device 200 onto the torso of the subject. The adjustment joints 1043J/1053J may be used to enable adjustment of intersect angle between two adjacent support sections 1043/1053, thereby to adjust distance between the two adjacent attachment disks 1042/1052 (e.g., the wider the intersect angle, the farther between the two adjacent attachment disks 1042/1053; the narrower the intersect angle, the closer between the two adjacent attachment disks 1042/1053).

In some embodiments, the connection relationships between the elements of the lead electrode support 104 (first lead electrode support) may be described as follows: the lead electrode support 104 may include a first support section (see the support section 1043 to the most left of FIG. 3A or FIG. 3B), a second support section (see the support section 1043 to the second left of FIG. 3A or FIG. 3B), a third support section (see the support section 1043 to the third left of FIG. 3A or FIG. 3B), a fourth support section (see the support section 1043 to the most right of FIG. 3A or FIG. 3B), a first adjustment joint (see the adjustment joint 1043J to the most left of FIG. 3A or FIG. 3B), a second adjustment joint (see the adjustment joint 1043J at middle of FIG. 3A or FIG. 3B), a third adjustment joint (see the adjustment joint 1043J to the most right of FIG. 3A or FIG. 3B), a first attachment disk (see the attachment disk 1042 to the most left of FIG. 3A or FIG. 3B), a second attachment disk (see the attachment disk 1042 to the middle of FIG. 3A or FIG. 3B), a third attachment disk (see the attachment disk 1042 to the most right of FIG. 3A or FIG. 3B) and a first connection knot (see the connection knot 1041 of FIG. 3A or FIG. 3B); the first support section and the second support section are pivotally connected through the first adjustment joint; the second support section and the third support section are pivotally connected through the second adjustment joint; the third support section and the fourth support section are pivotally connected through the third adjustment joint; the first attachment disk is coupled to free end of the first support section and may be used to attach to torso of the subject; the second attachment disk is coupled to the second adjustment joint and may be used to attach to torso of the subject; the third attachment disk is coupled to free end of the fourth support section and may be used to attach to torso of the subject; and the first connection knot is coupled to the first adjustment joint and may be used to couple the lead electrode support 104 to the branch support 102. With the above configurations of the connection knot 1041, the attachment disks 1042, the support sections 1043, and the adjustment joints 1043J, attachment positions for the lead electrode sensor device 200 on torso of the subject may be determined. For example, the first attachment disk of the three attachment disks 1042 may be used to indicate a fourth intercostal space on right margin of the sternum (first attachment position); the second attachment disk of the three attachment disks 1042 may be used to indicate a fourth intercostal space on left margin of the sternum (second attachment position); the third attachment disk of the three attachment disks 1042 may be used to indicate a midpoint between the fourth intercostal space on left margin of the sternum and a fifth intercostal space on the mid-clavicular line of the left clavicle (third attachment position).

In some other embodiments, similarly, the connection relationships between the elements of the lead electrode support 105 (second lead electrode support) may be described as follows: the lead electrode support 105 may include a fifth support section (see the support section 1053 to the most left of FIG. 3A or FIG. 3B), a sixth support section (see the support section 1053 to the second left of FIG. 3A or FIG. 3B), a seventh support section (see the support section 1053 to the third left of FIG. 3A or FIG. 3B), an eighth support section (see the support section 1053 to the most right of FIG. 3A or FIG. 3B), a fourth adjustment joint (see the adjustment joint 1053J to the most left of FIG. 3A or FIG. 3B), a fifth adjustment joint (see the adjustment joint 1053J at middle of FIG. 3A or FIG. 3B), a sixth adjustment joint (see the adjustment joint 1053J to the most right of FIG. 3A or FIG. 3B), a fourth attachment disk (see the attachment disk 1052 to the most left of FIG. 3A or FIG. 3B), a fifth attachment disk (see the attachment disk 1052 to the middle of FIG. 3A or FIG. 3B), a sixth attachment disk (see the attachment disk 1052 to the most right of FIG. 3A or FIG. 3B) and a second connection knot (see the connection knot 1051 of FIG. 3A or FIG. 3B); the fifth support section and the sixth support section are pivotally connected through the fourth adjustment joint; the sixth support section and the seventh support section are pivotally connected through the fourth adjustment joint; the seventh support section and the eighth support section are pivotally connected through the sixth adjustment joint; the fourth attachment disk is coupled to free end of the fifth support section and may be used to attach to torso of the subject; the fifth attachment disk is coupled to the fifth adjustment joint and may be used to attach to torso of the subject; the sixth attachment disk is coupled to free end of the eighth support section and may be used to attach to torso of the subject; and the second connection knot is coupled to the fourth adjustment joint and may be used to couple the lead electrode support 105 to the branch support 103. With the above configurations of the connection knot 1051, the attachment disks 1052, the support sections 1053, and the adjustment joints 1053J, attachment positions for the lead electrode sensor device 200 on torso of the subject may be determined. For example, the fourth attachment disk of the three attachment disks 1052 may be used to indicate the fifth intercostal space on the mid-clavicular line of the left clavicle (fourth attachment position); the fifth attachment disk of the three attachment disks 1052 may be used to indicate a fifth intercostal space on a left anterior axillary line of the subject (fifth attachment position); and the sixth attachment disk of the three attachment disks 1052 may be used to indicate a fifth intercostal space on a left mid-axillary line of the subject (sixth attachment position).

The above arrangement of the main support 101, the branch supports 102 and 103, and the lead electrode supports 104 and 105 with respect to the subject in standing posture may enable accurate placement of the lead electrode sensor device 200 according to heart within thoracic cavity of the subject, thereby to accurately collect electrocardiogram signal data corresponding to desired regions of the heart.

The positioning device 100 may further include measurement units 100S. The measurement units 100S are removably disposed along longitudinal directions of the main support 101, the branch support 102 and the branch support 103 respectively. The measurement units 100S may be realized as inertial measurement units (IMU) and/or electronic protractors to establish coordinate system for the subject once the positioning device 100 are attached to the torso of the subject. Although measurement units 100S are only shown on branch supports 102 and 103 in FIG. 2, other measurement units 100S may also be present according to requirements for establishing coordinate system for the subject. For example, IMUs and/or electronic protractors may be installed in each of the joint 101J, the sections 102S, the sections 103S, the attachment disks 1042/1052 and the adjustment joints 1043J/1053J, thereby to establish coordinate system that best describes positional information of thoracic cavity of the subject.

The lead electrode sensor device 200 may include lead electrodes 201 to 206 and a circuit layer 200C electrically connected with the lead electrodes 201 to 206. The lead electrodes 201 to 206 and the circuit layer 200C may be attached to the torso of the subject according to the attachment position determined by the lead electrode supports 104 and 105. Therefore, the modularized configuration of the lead electrode sensor device 200 may enable fast and precise attachment on to torso of the subject.

When placing the lead electrode sensor device 200 on the subject in standing posture using the positioning device 100, the arrangements of the lead electrodes 201 to 206 may be described as follows: the lead electrode 201 (first lead electrode) may be attached to the fourth intercostal space on right margin of the sternum (first attachment position) as indicated by the first attachment disk of the lead electrode support 104; the lead electrode 202 (second lead electrode) may be attached to the fourth intercostal space on left margin of the sternum (second attachment position) as indicated by the second attachment disk of the lead electrode support 104; the lead electrode 204 (fourth lead electrode) may be attached to fifth intercostal space on the mid-clavicular line of the left clavicle the subject (fourth attachment position) as indicated by the fourth attachment disk of the lead electrode support 105; the lead electrode 203 (third lead electrode) may be attached to midpoint between the lead electrode 202 and the lead electrode 204 (third attachment position) as indicated by the third attachment disk of the lead electrode support 104; the lead electrode 205 (fifth lead electrode) may be attached to the fifth intercostal space on left anterior axillary line of the subject (fifth attachment position) as indicated by the fifth attachment disk of the lead electrode support 105; the lead electrode 206 (sixth lead electrode) may be attached to the fifth intercostal space on left mid-axillary line of the subject (sixth attachment position) as indicated by the sixth attachment disk of the lead electrode support 105. When observed from the standing posture of the subject, the lead electrodes 204 to 206 attached on the subject is at a same height with respect to ground. The above arrangement of the lead electrodes 201 to 206 may enable collection of electrocardiogram signal data corresponding to desired regions of the heart within thoracic cavity of the subject.

The above arrangement of the lead electrodes 201 to 206 with respect to the subject in standing posture may enable accurate placement of the lead electrode sensor device 200 corresponding to heart within thoracic cavity of the subject, thereby to accurately collect electrocardiogram signal data corresponding to desired regions of the heart. Moreover, the lead electrode sensor device 200 may be made of elastic materials to enable adjustment according to variations of body types. For example, the spacing 11 between the lead electrodes 201 and 202, the spacing 12 between the lead electrodes 202 and 203, the spacing 13 between the lead electrodes 203 and 204, the spacing 14 between the lead electrodes 204 and 205 and the spacing 15 between the lead electrodes 205 and 206 may each has a stretching range for up to 0.8 cm. Therefore, if the subject has a wider chest figure compared to average human beings, the spacings 11 to 15 may be stretched and fixed by attachment disks 1042/1052 to maintain accurate placement of the lead electrodes 201 to 206 corresponding to desired regions of heart within thoracic cavity of the subject and without influence by the body shape of the subject.

The probe sensor device 300 may include at least one sensor probes 300P. The sensor probes 300P may be realized as ultrasound sensing probes and/or microphone arrays and are removably disposed along longitudinal direction of the branch support arm 102 and the branch support 103 respectively. The sensor probes 300P may be used to emit and retrieve ultrasound signals transmitting into and reflecting out of the body surface of the subject. The ultrasound signals may be configured in continuous wave signals or pulse wave signals. When the sensor probes 300P are disposed on body surface of the torso of the subject, a heart topology and relative positions between the heart and the respective sensor probes 300P may be detected. The relative positions between the heart and the sensor probes 300P may represent a distance from specific locations of body surface to the heart within the subject, and may be used to compute the heart coordinate within the coordinate system established by the measurement units 100S. The heart topology may be used to compute established digital twin of the heart. The established digital twin of the heart may be used to demonstrate ischemic condition of the subject according to electrocardiogram signal data detected by the lead electrode device 200.

The configurations of the positioning device 100, the lead electrode sensor device 200 and the probe sensor device 300 shown in FIG. 2, FIG. 3A, FIG. 3B, FIG. 4A and FIG. 4B may also be adjusted according operational requirements. For example, although only six measurement units 100S are shown in the positioning device 100, other inertial measurement units or electronic protractors may be disposed on the positioning device 100 to establish a more precise coordinate system for the subject. In another example, the modularized configuration of the lead electrode sensor device 200 may be adjusted to accommodate more lead electrodes 201 to 206 and a larger circuit layer 200C, so as to detect more variations of the electrocardiogram signal data from body surface of the subject and achieve a more precise computation for the ischemic condition for the subject. In a further example, although only four sensor probes 300P are shown in the probe sensor device 300, other ultrasound sensing probes and/or microphone arrays may be disposed on the positioning device 100 to construct a more precise established digital twin of the heart for the subject.

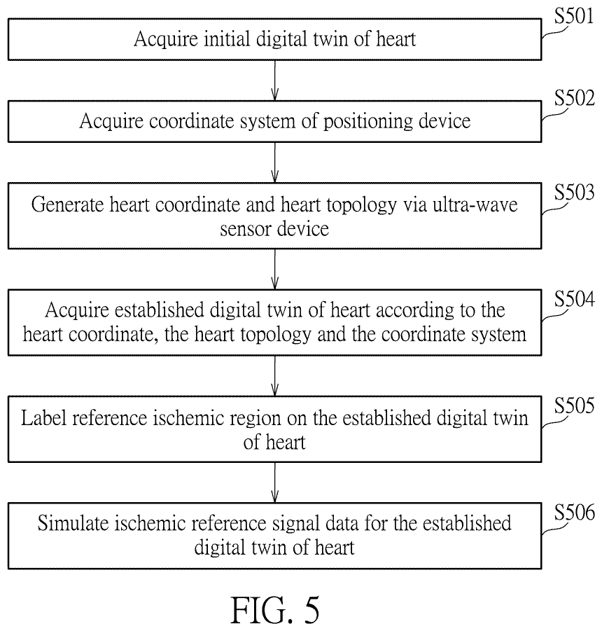

FIG. 5 is a flow diagram illustrating steps conducted by the analysis device 500 to compute an established digital twin of the heart of the subject and ischemic reference signal data for the established digital twin of the heart for virtual cardiac ischemia imaging. The steps S501 to S506 in FIG. 5 may be performed after the positioning device 100, the lead electrode sensor device 200 and the probe sensor device 300 are attached onto the subject according to the attachment position.

At step S501, the analysis device 500 may acquire an initial digital twin of a heart model from the database 400. The initial digital twin of the heart model may be a 257-node three-dimensional shape file of a heart stored in the database 400. The three-dimensional shape file of the heart may correspond to a general three-dimensional shape of a human heart based on computed tomography scan and/or magnetic resonance imaging of patients being admitted to a medical institution.

At step S502, the analysis device 500 may acquire the coordinate system established by the measurement units 100S of the positioning device 100 from the database 400.

At step S503, the analysis device 500 may acquire the relative position (e.g., relative positions detected by the sensor probes 300P) between body surface and heart of the subject and the heart topology detected by the probe sensor device 300 from the database 400 and further compute the heart coordinate of the heart of the subject within the coordinate system. The heart topology may be used to determine actual three-dimensional shape of the heart of the subject based on differences between the heart topology and the initial digital twin of the heart model.

At step S504, the analysis device 500 may compute the established digital twin of the heart according to the heart coordinate, the heart topology, the coordinate system and the initial digital twin of the heart model. The heart coordinate may be used to locate the established digital twin of the heart within the coordinate system. The heart topology and the initial digital twin of the heart model may be used to mold the established digital twin of the heart from the initial digital twin of the heart model according to actual three-dimensional shape of the heart of the subject. For example, the 257 nodes of the three-dimensional shape file of the heart model may be adjusted according to the heart topology to acquire the established digital twin of the heart of the subject.

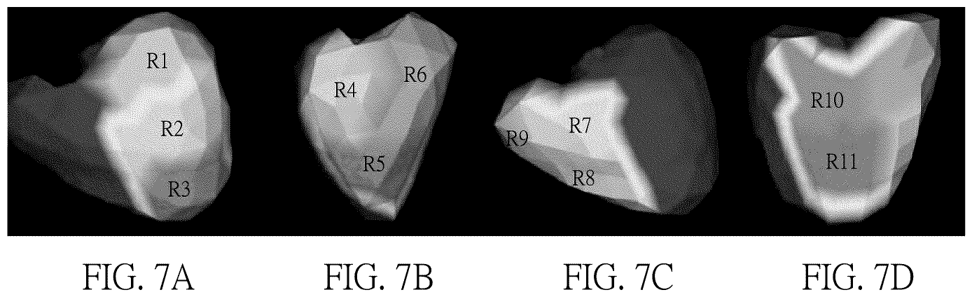

At step S505, with reference to FIGS. 7A to 7D, the analysis device 500 may further label reference ischemic region on the established digital twin of the heart of the subject. The reference ischemic regions, based on anatomical areas of the heart of the subject, may correspond to 26 modeled regions formed by 11 basic regions R1 to R11, which may be listed in table 1 below:

TABLE 1
reference ischemic regions

| | | | | | | | | | | | | Modeled |
|---|---|---|---|---|---|---|---|---|---|---|---|---|
| Infarcted | Eleven Basic Regions | | | | | | | | | | | region |
| Artery | R1 | R2 | R3 | R4 | R5 | R6 | R7 | R8 | R9 | R10 | R11 | No. |
| left | X | | | | | | | | | | | 1 |
| anterior | | X | | | | | | | | | | 2 |
| descending | | | X | | | | | | | | | 3 |
| | X | X | | | | | | | | | | 4 |
| | | X | X | | | | | | | | | 5 |
| | X | X | X | | | | | | | | | 6 |
| left | | | | X | | | | | | | | 7 |
| circumflex | | | | | X | | | | | | | 8 |
| coronary | | | | | | X | | | | | | 9 |
| artery | | | | X | X | | | | | | | 10 |
| | | | | | X | X | | | | | | 11 |
| | | | | X | | X | | | | | | 12 |
| | | | | X | X | X | | | | | | 13 |
| right | | | | | | | X | | | | | 14 |
| coronary | | | | | | | | X | | | | 15 |
| artery | | | | | | | | | X | | | 16 |
| | | | | | | | | | | X | | 17 |
| | | | | | | | | | | | X | 18 |
| | | | | | | | X | X | | | | 19 |
| | | | | | | | X | | X | | | 20 |
| | | | | | | | | X | X | | | 21 |
| | | | | | | | | | | X | X | 22 |
| | | | | | | | | | X | X | | 23 |
| | | | | | | | | | X | X | X | 24 |
| | | | | | | | X | X | X | | | 25 |
| | | | | | | | X | X | X | X | X | 26 |

The columns marked with “X” represent the basic regions that form the modeled regions.

At step S506, the analysis device 500 may simulate ischemic reference signal data according to the reference ischemic regions for the established digital twin of the heart of the subject. For example, one piece of the ischemic reference signal may be used to simulate one piece of electrocardiogram signal data corresponding to an ischemic level present at one of the 26 reference ischemic regions, where the ischemic level may range from level 1 to level 5. In another example, a specific reference ischemic region may be used to simulate 2ⁿ×5 pieces of ischemic reference signal data, where n represents number of nodes of the established digital twin of the heart contained in a that specific reference ischemic region contributing to an ischemic condition. In a further example, a complete set of the ischemic reference signal data may be categorized to 130 reference ischemic conditions based on the 26 reference ischemic regions and the 5 ischemic levels (i.e. 26×5=130 reference ischemic conditions). The ischemic reference signal data corresponding to the 130 categories of reference ischemic conditions may be stored in the database 400 and compared with the electrocardiogram signal data acquired by the lead electrode sensor device 200 at a later step to determine actual ischemic condition of the subject.

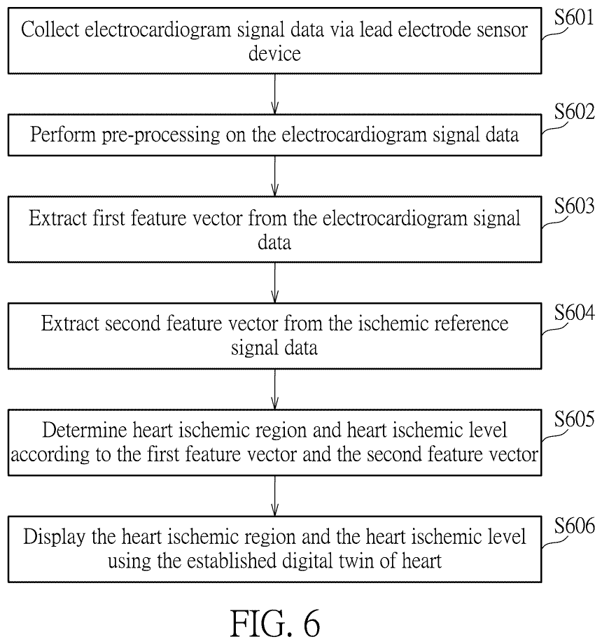

FIG. 6 is a flow diagram illustrating steps conducted by the analysis device 500 to determine ischemic condition of the subject using the established digital twin of the heart of the subject and the electrocardiogram signal data acquired by the lead electrode sensor device 200 during virtual cardiac ischemia imaging. The steps S601 to S606 in FIG. 6 may be performed after the established digital twin of the heart of the subject is computed and stored in the database 400.

At step S601, the analysis device 500 may collect the electrocardiogram signal data detected via lead electrodes 201 to 206 of the lead electrode sensor device 200. The electrocardiogram signal data may be stored in the database 400.

At step S602, the analysis device 500 may perform pre-processing on the electrocardiogram signal data to enhance signal quality of the electrocardiogram signal data before analysis. The pre-processing on the electrocardiogram signal data may include procedures such denoising, as normalization, resampling, segmentation, outlier removal, etc. performed on the electrocardiogram signal data.

At step S603, the analysis device 500 may extract first feature vector from the electrocardiogram signal data. For example, one piece of electrocardiogram signal data may correspond to a PORST wave of a cardiac cycle, and the first feature vectors may be derived from J point (i.e., intersecting point between QRS segment and ST segment), J−0.25×J−T length, J−0.5×J−T length, maximum amplitude of T wave and minimum amplitude of T wave in that piece of the electrocardiogram signal data.

At step S604, the analysis device 500 may extract second feature vector from the ischemic reference signal data. For example, one piece of ischemic reference signal data may correspond to a PORST wave of a simulated cardiac cycle, and the second feature vectors may be derived from simulated J point, simulated J−0.25×J−T length, simulated J−0.5×J−T length, maximum amplitude of simulated T wave and minimum amplitude of simulated T wave in that piece of the ischemic reference signal data.

At step S605, the analysis device 500 may determine the ischemic condition of the subject based on the first feature vector and the second feature vector, where the ischemic condition may indicate the heart ischemic region and the heart ischemic level of the heart ischemic region for the subject. For example, each piece of the ischemic reference signal data corresponds to a reference ischemic condition (e.g., one of the 130 categories of reference ischemic conditions) having a reference ischemic region and an ischemic level of the reference ischemic region, and the analysis device 500 may conduct a sparse representation classification (SRC) to compute similarity score between the first feature vector of the electrocardiogram signal data and the second feature vector of each piece of the ischemic reference signal data, determine one piece of the ischemic reference signal data having the second feature vector with highest similarity score with respect to the first feature vector, determine the reference ischemic condition corresponding to that one piece of the ischemic reference signal data as the ischemic condition of the subject, and set the reference ischemic region and the ischemic level described in that reference ischemic condition to be the heart ischemic region and heart ischemic level of the heart ischemic region for the subject, respectively.

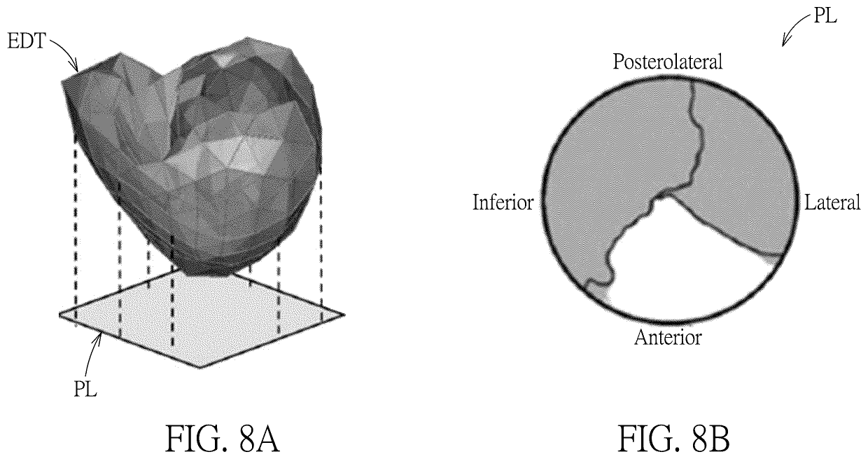

At step S606, as shown in FIGS. 8A and 8B, the analysis device 500 may instruct the display device 600 to display the ischemic condition on the established digital twin EDT of the heart of the subject on a two-dimensional plane PL. For example, the analysis device 500 may: label the heart ischemic region and the heart ischemic level of the heart ischemic region on the established digital twin EDT of the heart of the subject, compute projection of the established digital twin EDT of the heart of the subject, along with the heart ischemic region and the heart ischemic level of the heart ischemic region, onto the plane PL by referencing to the 257 nodes of the established digital twin EDT of the heart, and instruct the display device 600 to display the plane PL describing heart of the subject labeled with the heart ischemic region and the heart ischemic level. In here, by using the 257 nodes of the established digital twin EDT of the heart for computation of projection, positioning relationships of the heart ischemic region within the established digital twin EDT of the heart may be preserved on the plane PL.

Based on the above, the system and method for virtual cardiac ischemia imaging may be used to precisely attach lead electrodes on to body surface of torso of a subject in need thereof, precisely simulate ischemic condition of heart of the subject and does not require utilization of nuclear medicine examination to estimate shape of the heart of the subject. Therefore, time is saved for electrocardiographic imaging for the subject, human error for determining ischemic condition of the subject is reduced and concerns of subject being put under exposure to radiation may be avoided.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A system for virtual cardiac ischemia imaging, comprising:
a positioning device configured to position an attachment position and establish a coordinate system for a subject in need thereof;
a probe sensor device coupled with the positioning device and configured to acquire a relative position from body surface to a heart of the subject and a heart topology for the heart of the subject;
a lead electrode sensor device coupled with the positioning device and configured to acquire electrocardiogram signal data for the subject from the attachment position;
an analysis device coupled to the probe sensor device and the lead electrode sensor device and configured to:
establish an established digital twin of the heart according to an initial digital twin of a heart model, a heart coordinate according to the relative position within the coordinate system, the heart topology, and the coordinate system;
label a reference ischemic region on the established digital twin of the heart;
simulate ischemic reference signal data according to the reference ischemic region for the established digital twin of the heart;
perform pre-processing on the electrocardiogram signal data;
extract a first feature vector from the electrocardiogram signal data;
extract a second feature vector from the ischemic reference signal data;
determine a heart ischemic region of the heart and a heart ischemic level of the heart ischemic region according to the first feature vector and the second feature vector; and
label the heart ischemic region and the heart ischemic level of the heart ischemic region on the established digital twin of the heart; and
a display device coupled to the analysis device and configured to display the heart ischemic region and the heart ischemic level of the heart ischemic region on the established digital twin of the heart on a two-dimensional plane.

2. The system of claim 1, wherein the positioning device comprises:
a main support placed along a longitudinal direction of a left clavicle of the subject;
a first branch support hingedly connected to the main support and placed along a longitudinal direction of a sternum of the subject;
a second branch support hingedly connected to the main support and placed along a longitudinal direction of a mid-clavicular line of the left clavicle of the subject;
a first lead electrode support removably connected to the first branch support and branch from the first branch support to a torso surface of the subject; and
a second lead electrode support removably connected to the second branch support and branch from the second branch support to the torso surface of the subject.

3. The system of claim 2, wherein:
the first lead electrode support comprises:
a first support section;
a second support section pivotally connected with the first support section through a first adjustment joint;
a third support section pivotally connected with the second support section through a second adjustment joint;
a fourth support section pivotally connected with the third support section through a third adjustment joint;
a first attachment disk coupled to a free end of the first support section;
a second attachment disk coupled to the second adjustment joint; and
a third attachment disk coupled to a free end of the fourth support section;
the first attachment disk indicating a fourth intercostal space on a right margin of the sternum;
the second attachment disk indicating a fourth intercostal space on a left margin of the sternum; and
the third attachment disk indicating a midpoint between the fourth intercostal space on the left margin of the sternum and a fifth intercostal space on the mid-clavicular line of the left clavicle; and
the second lead electrode support comprises:
a fifth support section;
a sixth support section pivotally connected with the fifth support section through a fourth adjustment joint;
a seventh support section pivotally connected with the sixth support section through a fifth adjustment joint;
an eighth support section pivotally connected with the seventh support section through a sixth adjustment joint;
a fourth attachment disk coupled to a free end of the fifth support section;
a fifth attachment disk coupled to the fifth adjustment joint; and
a sixth attachment disk coupled to a free end of the eighth support section;
the fourth attachment disk indicating the fifth intercostal space on the mid-clavicular line of the left clavicle;
the fifth attachment disk indicating a fifth intercostal space on a left anterior axillary line of the subject; and
the sixth attachment disk indicating a fifth intercostal space on a left mid-axillary line of the subject.

4. The system of claim 3, wherein:
the attachment position comprises a first attachment position, a second attachment position, a third attachment position, a fourth attachment position, a fifth attachment position, and a sixth attachment position;
the lead electrode sensor device comprises:
a first lead electrodes attached to the first attachment position, wherein the first attachment position corresponds to the fourth intercostal space on the right margin of the sternum indicated by the first attachment disk;
a second lead electrodes attached to the second attachment position, wherein the second attachment position corresponds to the fourth intercostal space on the left margin of the sternum indicated by the second attachment disk;
a fourth lead electrodes attached to the fourth attachment position, wherein the fourth attachment position corresponds to the fifth intercostal space on the mid-clavicular line of the left clavicle indicated by the fourth attachment disk;
a third lead electrodes attached to the third attachment position, wherein the third attachment position corresponds to the midpoint between the second lead electrode and the fourth lead electrode indicated by the third attachment disk;
a fifth lead electrodes attached to the fifth attachment position, wherein the fifth attachment position corresponds to the fifth intercostal space on the left anterior axillary line indicated by the fifth attachment disk; and
a sixth lead electrodes attached to the sixth attachment position, wherein the sixth attachment corresponds to the fifth intercostal space on the left mid-axillary line of the subject indicated by the sixth attachment disk.

5. The system of claim 3, wherein:
the main support comprises at least one joint;
the first branch support is made of at least two first sections hingedly concatenated in a row;
the second branch support is made of at least two second sections hingedly concatenated in a row;
the positioning device further comprises at least one measurement unit disposed in: at least one of the at least two first sections, at least one of the two second sections, the first attachment disk, the second attachment disk, the third attachment disk, the fourth attachment disk, the fifth attachment disk, the sixth attachment disk, the first adjustment joint, the second adjustment joint, the third adjustment joint, the fourth adjustment joint, the fifth adjustment joint, and/or the sixth adjustment joint; and
the measurement unit is an inertial measurement unit or an electronic protractor.

6. The system of claim 2, wherein:
the probe sensor device comprises at least one sensor probe removably disposed along longitudinal direction of the first branch support and/or along longitudinal direction of the second branch support;
the analysis module is further configured to compute the heart coordinate within the coordinate system using the relative position; and
the sensor probe is an ultrasound sensing probe or a microphone array.

7. The system of claim 1, wherein:
each piece of the ischemic reference signal data corresponds to a reference ischemic condition having the reference ischemic region and an ischemic level; and
the analysis device determining heart ischemic region of the heart and heart ischemic level of the heart ischemic region according to the first feature vector and the second feature vector comprises:
conducting a sparse representation classification to compute a similarity score between the first feature vector and the second feature vector of each piece of the ischemic reference signal data;
determining one piece of the ischemic reference signal data having the second feature vector with highest similarity score with respect to the first feature vector;
determining the reference ischemic condition corresponding to the one piece of the ischemic reference signal data as an ischemic condition of the subject; and
setting the reference ischemic region and the ischemic level described in the reference ischemic condition to be the heart ischemic region and heart ischemic level of the heart ischemic region for the subject, respectively.

8. The system of claim 1, wherein the analysis device is further configured to:
compute projection of the established digital twin, the heart ischemic region, and the heart ischemic level of the heart ischemic region onto the two-dimensional plane.

9. A method for virtual cardiac ischemic imaging, comprising:
a positioning device positioning an attachment position and establishing a coordinate system for a subject in need thereof;
a probe sensor device acquiring a relative position from body surface to a heart of the subject and a heart topology for the heart of the subject;
a lead electrode sensor device acquiring electrocardiogram signal data for the subject from the attachment position;
an analysis device establishing an established digital twin of the heart according to an initial digital twin of a heart model, a heart coordinate according to the relative position within the coordinate system, the heart topology and the coordinate system;
the analysis device labeling a reference ischemic region on the established digital twin of the heart;
the analysis device simulating ischemic reference signal data according to the reference ischemic region for the established digital twin of the heart;
the analysis device performing pre-processing on the electrocardiogram signal data;
the analysis device extracting a first feature vector from the electrocardiogram signal data;
the analysis device extracting a second feature vector from the ischemic reference signal data;
the analysis device determining a heart ischemic region of the heart and a heart ischemic level of the heart ischemic region according to the first feature vector and the second feature vector;
the analysis device labeling the heart ischemic region and the heart ischemic level of the heart ischemic region on the established digital twin of the heart; and
a display device displaying the heart ischemic region and the heart ischemic level of the heart ischemic region on the established digital twin of the heart on a two-dimensional plane.

10. The method of claim 9, wherein the positioning device comprises:
a main support placed along a longitudinal direction of a left clavicle of the subject;
a first branch support hingedly connected to the main support and placed along a longitudinal direction of a sternum of the subject;
a second branch support hingedly connected to the main support and placed along a longitudinal direction of a mid-clavicular line of the left clavicle of the subject;
a first lead electrode support removably connected to the first branch support and branch from the first branch support to a torso surface of the subject; and
a second lead electrode support removably connected to the second branch support and branch from the second branch support to the torso surface of the subject.

11. The method of claim 10, wherein:
the first lead electrode support comprises:
a first support section;
a second support section pivotally connected with the first support section through a first adjustment joint;
a third support section pivotally connected with the second support section through a second adjustment joint;
a fourth support section pivotally connected with the third support section through a third adjustment joint;
a first attachment disk coupled to a free end of the first support section;
a second attachment disk coupled to the second adjustment joint; and
a third attachment disk coupled to a free end of the fourth support section;
the first attachment disk indicating a fourth intercostal space on a right margin of the sternum;
the second attachment disk indicating a fourth intercostal space on a left margin of the sternum; and
the third attachment disk indicating a midpoint between the fourth intercostal space on the left margin of the sternum and a fifth intercostal space on the mid-clavicular line of the left clavicle; and
the second lead electrode support comprises:
a fifth support section;
a sixth support section pivotally connected with the fifth support section through a fourth adjustment joint;
a seventh support section pivotally connected with the sixth support section through a fifth adjustment joint;
an eighth support section pivotally connected with the seventh support section through a sixth adjustment joint;
a fourth attachment disk coupled to a free end of the fifth support section;
a fifth attachment disk coupled to the fifth adjustment joint; and
a sixth attachment disk coupled to a free end of the eighth support section;
the fourth attachment disk indicating the fifth intercostal space on the mid-clavicular line of the left clavicle;
the fifth attachment disk indicating a fifth intercostal space on a left anterior axillary line of the subject; and
the sixth attachment disk indicating a fifth intercostal space on a left mid-axillary line of the subject.

12. The method of claim 11, wherein:
the attachment position comprises a first attachment position, a second attachment position, a third attachment position, a fourth attachment position, a fifth attachment position and a sixth attachment position;
the lead electrode sensor device comprises:
a first lead electrodes attached to the first attachment position, wherein the first attachment position corresponds to the fourth intercostal space on the right margin of the sternum indicated by the first attachment disk;
a second lead electrodes attached to the second attachment position, wherein the second attachment position corresponds to the fourth intercostal space on the left margin of the sternum indicated by the second attachment disk;
a fourth lead electrodes attached to the fourth attachment position, wherein the fourth attachment position corresponds to the fifth intercostal space on the mid-clavicular line of the left clavicle indicated by the fourth attachment disk;
a third lead electrodes attached to the third attachment position, wherein the third attachment position corresponds to the midpoint between the second lead electrode and the fourth lead electrode indicated by the third attachment disk;
a fifth lead electrodes attached to the fifth attachment position, wherein the fifth attachment position corresponds to the fifth intercostal space on the left anterior axillary line indicated by the fifth attachment disk; and
a sixth lead electrodes attached to the sixth attachment position, wherein the sixth attachment corresponds to the fifth intercostal space on the left mid-axillary line of the subject indicated by the sixth attachment disk.

13. The method of claim 11, wherein:
the main support comprises at least one joint;
the first branch support is made of at least two first sections hingedly concatenated in a row;
the second branch support is made of at least two second sections hingedly concatenated in a row;
the positioning device further comprises at least one measurement unit disposed in: at least one of the at least two first sections, at least one of the two second sections, the first attachment disk, the second attachment disk, the third attachment disk, the fourth attachment disk, the fifth attachment disk, the sixth attachment disk, the first adjustment joint, the second adjustment joint, the third adjustment joint, the fourth adjustment joint, the fifth adjustment joint and/or the sixth adjustment joint and
the at least one measurement unit is an inertial measurement unit or an electronic protractor.

14. The method of claim 10, wherein:
the probe sensor device comprises at least one sensor probe removably disposed along longitudinal direction of the first branch support and/or along longitudinal direction of the second branch support;
the method further comprises the analysis module computing the heart coordinate within the coordinate system using the relative position; and
the at least one sensor probe is an ultrasound sensing probe or a microphone array.

15. The method of claim 9, wherein:
each piece of the ischemic reference signal data corresponds to a reference ischemic condition having the reference ischemic region and an ischemic level of the reference ischemic region; and
the analysis device determining the heart ischemic region of the heart and the heart ischemic level of the heart ischemic region according to the first feature vector and the second feature vector comprises:
the analysis device conducting a sparse representation classification to compute a similarity score between the first feature vector and the second feature vector of each piece of the ischemic reference signal data;
the analysis device determining one piece of the ischemic reference signal data having the second feature vector with highest similarity score with respect to the first feature vector;
the analysis device determining the reference ischemic condition corresponding to the one piece of the ischemic reference signal data as an ischemic condition of the subject; and
the analysis device setting the reference ischemic region and the ischemic level described in the reference ischemic condition to be the heart ischemic region and heart ischemic level of the heart ischemic region for the subject, respectively.

16. The method of claim 9, further comprising:
the analysis device computing projection of the established digital twin, the heart ischemic region and the heart ischemic level of the heart ischemic region onto the two-dimensional plane.
